(19) United States
(12) Patent Application Publication
(10) Pub. No.: US 2026/0097175 A1
(43) Pub. Date: Apr. 9, 2026

(54) Lung-Selectable Endotracheal Aspiration System and Methods for Draining a User-Selected Lung

(71) Applicant: Roberts Innovations, LLC, Delray Beach, FL (US)

(72) Inventors: John R. Roberts, Delray Beach, FL (US); Timothy D. Roberts, Delray Beach, FL (US)

(73) Assignee: Roberts Innovations, LLC, Delray Beach, FL (US)

(21) Appl. No.: 19/344,386

(22) Filed: Sep. 29, 2025

Related U.S. Application Data
(60) Provisional application No. 63/704,601, filed on Oct. 8, 2024.

(51) Int. Cl. A61M 16/04; A61M 16/20

(52) U.S. Cl. CPC A61M 16/0463; A61M 16/0434; A61M 16/20; A61M 2210/1032; A61M 2210/1039

(57) ABSTRACT

An endotracheal lung suction system includes an endotracheal tube defining a tube lumen, a suction assembly, and an inner suction catheter. The endotracheal tube comprises an inflation connector and an implantation balloon adjacent the distal tube end and fluidically connected to the inflation connection and through which the balloon inflates. The suction assembly comprises a proximal valve and a distal endotracheal tube connector fluidically connected to an interior of the tube lumen. The inner suction catheter passes through and slides within the suction assembly, the endotracheal tube connector, and through the tube lumen to extend slidably out from and back into the distal tube end. The inner suction catheter comprises a distal, inflatable, hollow, inner-suction-catheter securing device and a catheter body defining a main lumen extending through the inner suction catheter and a catheter-securing inflation lumen extending through the inner suction catheter parallel to the main lumen.

### CROSS-REFERENCE TO RELATED APPLICATIONS

This application claims the priority, under 35 U.S.C. § 119, of copending U.S. Provisional Ser. No. 63/704,601 , filed Oct. 8, 2024; the prior application is herewith incorporated by reference herein in its entirety.

### STATEMENT REGARDING FEDERALLY SPONSORED RESEARCH OR DEVELOPMENT

Not Applicable

### FIELD OF THE INVENTION

The present systems, apparatuses, and methods lie in the field of medical devices. The present disclosure relates to an endotracheal aspiration system that allows the surgeon to select into which lung (left or right) the distal end of the lung suction lumen is inserted and to close off the lung into which the suction tube is inserted for independent suction and/or aspiration.

### BACKGROUND OF THE INVENTION

The respiratory tract is an example of a tortuous pathway. The respiratory tract begins at the nose and mouth, which open to the trachea. The trachea travels downward into the chest and splits into the left and right main bronchi. The left and right main bronchi split at an angle from the trachea. The left main bronchus is smaller in diameter and branches at a greater angle from the trachea than the right main bronchus. The main bronchi then split into lobar bronchi, which split into segmental bronchi. The segmental bronchi split into subsegmental bronchi.

Several procedures require intubation of the respiratory tract, including the left and right main bronchi, to aspirate mucus from the airway or to deliver drugs to a specific airway. Intubation of the left main bronchus from the trachea can be difficult because it has a smaller diameter and greater angle relative to the trachea. For example, a typical procedure for aspirating fluid from the lungs includes introducing an endotracheal tube to the trachea of a patient, followed by extending a working catheter (e.g., an aspiration catheter) through a lumen of the endotracheal tube. Such a catheter almost always enters a single airway —usually the right airway. Respiratory therapists seeking to intubate the left main bronchus with the aspiration catheter may mistakenly believe the left main bronchus has been intubated, when the catheter actually has entered the right main bronchus instead. In some instances, the endotracheal tube can be mistakenly inserted too deep so that its distal end extends into the right main bronchus, whereby the aspiration catheter can only access the right main bronchus. Often times, a specialist, such as a pulmonologist, is needed to insert a bronchoscope into the left main bronchus and aspirate the left main bronchus using the working channel of the bronchoscope. The bronchoscope is equipped with a vision system (including, for example, a fiberoptic system) and/or a fluoroscopic imaging system, to guide the bronchoscope into the left main bronchus. However, visualization equipment and the endoscopic procedure is expensive, and specialists may not be readily available to conduct the procedure when desired.

Suction catheters are used to remove respiratory secretions and other material from airways and, in general, for treating or preventing a number of respiratory conditions, such as mucous plugging, bronchitis, and ventilator associated pneumonias. One exemplary treatment includes when patients develop pneumonia or bronchitis. With this condition, the patient coughs to clear the airways. If, however, the pneumonia worsens enough to require intubation with an endotracheal tube and placement on a ventilator (breathing machine), patients are unable to cough (due to sedation and the mechanical impediment of the endotracheal tube). Therefore, suction catheters are passed into the endotracheal tube and are employed to clear the infected mucous and, thus, improve the ability to breathe and help treat the infection. Suction catheters may also be used in patients without pneumonia to prevent the occurrence of pneumonia or other respiratory complications. It is known that the right bronchus (airway) is straighter and of greater diameter than the left. Accordingly, suction catheters passed into the trachea typically travel into the right bronchus more than 98% of the time. This anatomic fact is well known to physicians (pulmonologists, surgeons, and anesthesiologists) who manage the airway.

Closed system suction catheter assemblies used for removing secretions from within the trachea or bronchi of an intubated patient comprise a flexible catheter connected at its distal end to the proximal end of an endotracheal tube. The proximal end of the flexible catheter is connected with a fitting, including a valve, that can be opened or closed to control the application of suction to the catheter. The valve is usually of a kind having a flow control positioned lateral to the flow path and having two distinct positions where flow is either enabled or disabled.

Towards its distal end, the catheter extends through a fitting connected between the end of a tracheal tube and a ventilation circuit. The catheter can be advanced through the fitting down the tracheal tube to enable suctioning. A flexible envelope extends between the two couplings, enclosing the catheter so that it can be manipulated through the envelope. A wiper seal in the forward coupling prevents gas from the ventilation system inflating the envelope.

In some assemblies, provision is made for cleaning the catheter after its patient end has been withdrawn into the forward coupling. A manually-operable valve is located forward of the wiper seal providing a cleaning chamber between the valve and the wiper seal. An irrigation port opens into this chamber so that saline can be supplied to it, which is then drawn along the bore of the catheter by the applied suction to remove matter collected within the bore.

Some prior art suction catheters are passed blindly into the trachea and cannot be directed into either side. They are connected to the endotracheal tube and are kept on the patient's bed inside a sleeve that is not sterilized, and allow the bacteria to grow and accumulate. Thus, the catheters become contaminated, contaminate the sleeve, and re-introduce the same bacteria back into the patient's airway when suctioning is repeated. These catheters, therefore, can re-introduce the problem that they are designed to eradicate: infected secretions.

Because usually only the right lung is cleared of secretions, the left lung becomes a reservoir of infection, even if the right lung is the source of infection, as secretions from either lung move or contaminate the opposite lung. If the right lung is the source of the pneumonia, for example, this reservoir may be limited. However, if the left lung is the source, or becomes the secondary source, it will never be cleared by standard suctioning, and often requires bronchoscopy. This failure to clear the lung prolongs time on the ventilator, prolongs the recovery time from pneumonia, and increases the risk of developing resistant infections and of dying from pneumonia.

Together, pneumonia and influenza represented a cost to the U.S. economy in 2005 of $40.2 billion, $6 billion due to indirect mortality I costs and $34.2 billion in direct II costs, according to the American Lung Association. According to preliminary CDC mortality data from 2011, age-adjusted death rates decreased significantly from 2010 to 2011 for five of the fifteen leading causes of death (heart diseases, malignant neoplasms, cerebrovascular disease, Alzheimer's disease, and kidney diseases). However, the age-adjusted death rate increased for six leading causes of death: chronic lower respiratory diseases, diabetes mellitus, influenza and pneumonia, chronic liver disease and cirrhosis, Parkinson's disease, and pneumonitis due to solids and liquids. Three of these causes (chronic lower respiratory disease, influenza and pneumonia, and pneumonitis) are all variants of pneumonia. This data demonstrates that pneumonia is an already dangerous disease that is becoming more deadly.

U.S. Pat. No. 9,149,592 to Roberts et al. (hereinafter the “'592 Patent”) describes aspiration catheters, systems, and methods for draining lungs of a patient. FIGS. 1 to 4 of the '592 Patent show a catheter system with endotracheal tube having two internal drainage catheters or tubes that emerge from the distal end of the endotracheal tube. A user is able to manipulate one or both of the inner catheters to adjust the position or extend one or both of the inner catheters, in theory, into respective ones of the left and right main stems and, thereafter, into the respective left and right bronchus. The inner catheters are shown and described with bends at the distal ends that curve in opposite directions to create a V-or Y-shape. The inner catheters have a cross-sectional D-shape, with one side of the catheter being curved and the other side being flat. See, e.g., FIGS. 2A and 10. The inner catheters key into respectively shaped holes in a double key joint fitting within a ventilation adapter portion as shown in FIGS. 3A and 3B. The key joint fitting and the inner catheters, with the distal end curves and cross-sectional shapes, are costly to manufacture. FIG. 5 of the '592 Patent shows the catheter system in which a single inner catheter is disposed. FIGS. 7A and 7B show that single inner catheter and the distal single key joint fitting. Regardless of the shape of the inner catheters, it is difficult to accurately insert, either in the one-inner-catheter embodiment or the two-inner-catheter embodiment the selected inner catheter into the desired main stem. The two-inner-catheter embodiment provides a particularly disadvantageous configuration for a few reasons. First, the one inner catheter not being used (e.g., extended, retracted, and/or turned) interferes with the extension, retraction, and/or turning of the other inner catheter being used by the physician - it rests against the one inner catheter. This interference is sufficiently significant to have the physician forgo using the two-inner-catheter embodiment all together and only employ a system having the one-inner-catheter embodiment. Second, experience has shown that the Y-configuration of the two inner catheters is insufficient to guarantee placement of the adjacent two distal ends into two different main stems; in other words, there is a high probability that both distal ends enter the same main stem. It is not easy to determine if the inner catheter(s) is(are) correctly placed and, often, a bronchoscope is necessary to visualize placement of the distal ends of the inner catheters before use of either or both. The cost to manufacture the inner catheter embodiments of the '592 patent are not insignificant. To enable the turning feature described in the '592 patent, the inner catheter(s) costs more to produce than a simple circular cross-sectional catheter. It would be beneficial to not have to manufacture the inner catheter with such an asymmetric cross-section (flat/curved as in FIG. 10 or oval as in FIG. 12) and further beneficial if the inner catheter was a standardized suction tube.

A bronchial blocker (also called an endobronchial blocker) is a device that can be inserted down a tracheal tube after tracheal intubation to block off the right or left main bronchus of the lungs. This blocking allows a surgeon to achieve a controlled one-sided ventilation of the lungs during thoracic surgery. The lung tissue distal to the obstruction by the bronchial blocker will collapse, thereby allowing the surgeon view and access to relevant structures within the thoracic cavity. Bronchial blockers, however, are difficult to deploy because they must be guided with a separate guiding device, a bronchoscope, and even then cannot be placed reliably. Currently approved blockers require bronchoscopy to confirm placement

Further, double endotracheal tubes, like the one depicted in FIGS. 1 to 4B and 9A to 9E of the '592 Patent, typically catch upon the tracheal bifurcation and do not, with surety, place one of the tubes in each of the left and right main bronchus. Also, with double endotracheal tubes, use of one of the two suction catheters is inhibited by the rubbing that occurs against the other suction catheter not be used. This friction or rubbing prevents accurate placement of the suction catheter being extended further into a lung. Finally, the cost to produce these tubes is greater than the cost of a blocker. Because of these significant disadvantages, most surgeons would avoid use of double endotrachial tubes entirely if there were a feasible alternative that was easy to place.

A need exists to overcome the problems with the prior art systems, designs, and processes as discussed above. There exists a need for improved treatment and prevention of pneumonia and other respiratory conditions and complications. There exists a need for a bronchial blocker that can be easily placed.

### SUMMARY OF THE INVENTION

The systems, apparatuses, and methods described provide a lung-selectable endotracheal aspiration system and methods for draining a user-selected lung that overcome the hereinafore-mentioned disadvantages of the heretofore-known devices and methods of this general type and that provide such features with a guarantee of insertion into the lung that the surgeon-user desires.

Having the inner catheter securing and blocking device (e.g., a balloon) installed on the actual suction catheter allows the bronchus in which the suction tube is disposed (left main stem/right main stem) to be fluidically cut off from ventilation of that lung and only supply ventilation to the lung that is not being drained, for example.

As used herein, inhalation refers to the delivery of both gas and liquid and aspiration refers to the retraction or removal of both gas and liquid. The aspiration delivery systems and methods of the instant application are used to deliver gas to one or more lungs for patient respiration. Gas can be in the form of air or oxygen, to name a few.

The devices, systems, and methods of the instant application are not limited to suction; they can be both a withdrawal and a delivery device for liquid/material therethrough. The systems and methods of the instant application are further used to deliver fluid to one or more lungs for patient treatment. For example, liquid can be used to lavage the cavities in one or both of the lungs in which at least a portion of the systems of the instant application are deployed. As another example, medicinal liquid (such as antibiotics, antiseptics, and/or anti-inflammatory agents) can be used to medically or chemically treat the cavities of the lungs in which at least a portion of the systems of the instant application are used.

The aspiration removal systems and methods of the instant application are used to remove fluid from one or both lungs of a patient, e.g., to treat pulmonary edema. The fluid to be removed from one or more of the lungs can be in the form of mucus or aspirated gastric contents.

With the foregoing and other objects in view, there is provided, an endotracheal lung suction system comprising an endotracheal tube defining a tube lumen and comprising a distal tube end defining a distal opening of the tube lumen, a proximal tube end defining a proximal opening of the tube lumen, an inflation connector, and an implantation balloon fluidically connected to the inflation connection and through which the implantation balloon is inflated, the implantation balloon disposed adjacent the distal tube end, a suction assembly comprising a distal endotracheal tube connector at the proximal tube end and defining a tube opening fluidically connected to an interior of the tube lumen through the distal opening of the tube lumen and a proximal valve defining a proximal valve catheter opening fluidically connected through the suction assembly to the interior of the tube lumen, and an inner suction catheter shaped and sized to pass through and slide within the suction assembly from proximal of the proximal valve, distally through the tube opening of the endotracheal tube connector, and through the tube lumen to extend slidably out from and back into the proximal opening of the distal tube end, the inner suction catheter comprising a proximal portion comprising a proximal catheter end, a distal portion comprising a distal catheter end, an inflatable, hollow, inner-suction-catheter securing device at the distal portion and comprising an inflation interior defining an inflation interior opening, and a catheter body defining a main lumen extending through the inner suction catheter from the proximal catheter end to the distal catheter end and a catheter-securing inflation lumen extending through the inner suction catheter parallel to the main lumen from the proximal portion distally to the inflation interior opening.

In accordance with another feature, there is provided an inner catheter steering line shaped and sized to pass through and slide within the main lumen of the inner suction catheter, the inner catheter steering line comprising a distal portion with a steering curve.

In accordance with a further feature, the steering curve comprises a given shape and at least the distal catheter end of the inner suction catheter is flexible such that placement of the steering curve within the distal end causes the distal catheter end to take approximately the given shape of the steering curve therewithin.

In accordance with an added feature, the given shape is an S-bend.

In accordance with an additional feature, the steering curve comprises a given shape and the inner suction catheter is flexible to permit the given shape of the steering curve to slide through the main lumen from the proximal catheter end to the distal catheter end.

In accordance with yet another feature, the given shape is an S-bend and the inner suction catheter is flexible to permit the S-bend of the steering curve to slide through the main lumen from the proximal catheter end to the distal catheter end and, responsive to the steering curve being placed in the distal catheter end, the distal catheter end takes approximately the S-bend shape.

In accordance with yet a further feature, the distal portion at the distal catheter end is curved.

In accordance with yet an added feature, the distal portion at the distal catheter end has an S-shape.

In accordance with a concomitant feature, the inner suction catheter further comprises a valve at the proximal portion, the valve permitting a leak-free, fluidic connection from the environment to the interior of the main lumen of the inner suction catheter.

Although the systems, apparatuses, and methods are illustrated and described herein as embodied in a user-selectable endotracheal lung suction system, it is, nevertheless, not intended to be limited to the details shown because various modifications and structural changes may be made therein without departing from the spirit of the invention and within the scope and range of equivalents of the claims. Additionally, well-known elements of exemplary embodiments will not be described in detail or will be omitted so as not to obscure the relevant details of the systems, apparatuses, and methods.

Additional advantages and other features characteristic of the systems, apparatuses, and methods will be set forth in the detailed description that follows and may be apparent from the detailed description or may be learned by practice of exemplary embodiments. Still other advantages of the systems, apparatuses, and methods may be realized by any of the instrumentalities, methods, or combinations particularly pointed out in the claims.

Other features that are considered as characteristic for the systems, apparatuses, and methods are set forth in the appended claims. As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one of ordinary skill in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the systems, apparatuses, and methods of the invention that are regarded as novel, it is believed that the systems, apparatuses, and methods will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the systems, apparatuses, and methods. Advantages of embodiments of the systems, apparatuses, and methods will be apparent from the following detailed description of the exemplary embodiments thereof, which description should be considered in conjunction with the accompanying drawings in which:

FIG. 1 is a side elevational view of an exemplary embodiment of an endotracheal lung suction system having an endotracheal tube with an implantation balloon in a deflated state and with an exemplary embodiment of a single inner suction catheter having an inner catheter securing and blocking device in a contracted, reduced, or unexpanded state, the inner suction catheter in a system installation position or initial state;

FIG. 2 is a fragmentary, partially cross-sectional, partially side elevational view of the endotracheal lung suction system of FIG. 1 inserted in a patient's mouth, esophagus, and trachea, the implantation balloon of the endotracheal tube in the deflated state and the inner suction catheter in system installation position in the initial state;

FIG. 3 is a fragmentary, partially cross-sectional, partially side elevational view of the endotracheal lung suction system of FIG. 2 with the implantation balloon of the endotracheal tube expanded within a patient's trachea in an inflated state and the inner suction catheter in a first deployed state with the inner catheter securing and blocking device in the unexpanded state;

FIG. 4 is an elevational view of an exemplary embodiment of a steering line for the endotracheal lung suction system of FIG. 1;

FIG. 5 is a fragmentary, partially cross-sectional, partially side elevational view of the endotracheal lung suction system of FIG. 3 with the implantation balloon of the endotracheal tube expanded within a patient's trachea in the inflated state, the inner suction catheter in the first deployed state with the inner catheter securing and blocking device in the unexpanded state, and an inner tube control guidewire in a deployed state within the inner suction catheter and positioned to place the distal end of the inner suction catheter in an entrance of a left main stem of a patient's lung;

FIG. 6 is a fragmentary, partially cross-sectional, partially side elevational view of the endotracheal lung suction system of FIG. 3 with the implantation balloon of the endotracheal tube expanded within a patient's trachea in the inflated state, the inner suction catheter in the first deployed state with the inner catheter securing and blocking device in the unexpanded state, and the inner tube control guidewire in a deployed state within the inner suction catheter and rotated to place the distal end of the inner suction catheter in an entrance of a right main stem of the patient's lung;

FIG. 7 is a fragmentary, partially cross-sectional, partially side elevational view of the endotracheal lung suction system of FIG. 6 with the inner suction catheter in a second deployed state further in the right main stem of the patient's lung with the inner tube control guidewire therein in the deployed state and with the inner catheter securing and blocking device in the unexpanded state;

FIG. 8 is a fragmentary, partially cross-sectional, partially side elevational view of the endotracheal lung suction system of FIG. 6 with the inner suction catheter in a third deployed state further in the right main stem of the patient's lung with the inner tube control guidewire therein in the deployed state and with the inner catheter securing and blocking device in the unexpanded state;

FIG. 9 is a fragmentary, partially cross-sectional, partially side elevational view of the endotracheal lung suction system of FIG. 8 with the inner suction catheter in the third deployed state, with the inner tube control guidewire entirely removed from the inner suction catheter, and with the inner catheter securing and blocking device in the unexpanded state;

FIG. 10 is a fragmentary, partially cross-sectional, partially side elevational view of the endotracheal lung suction system of FIG. 9 with the inner suction catheter in the third deployed state and with an exemplary embodiment of the inner catheter securing and blocking device in an expanded or enlarged state;

FIG. 11 is an enlarged, cross-sectional view of an exemplary embodiment of a suction catheter;

FIG. 12 is a fragmentary, enlarged, perspective view of an exemplary embodiment of a inner catheter securing and blocking device; and FIG. 13 is a block diagram of an exemplary embodiment of a method of performing a procedure with the endotracheal lung suction system of FIGS. 1 and 4.

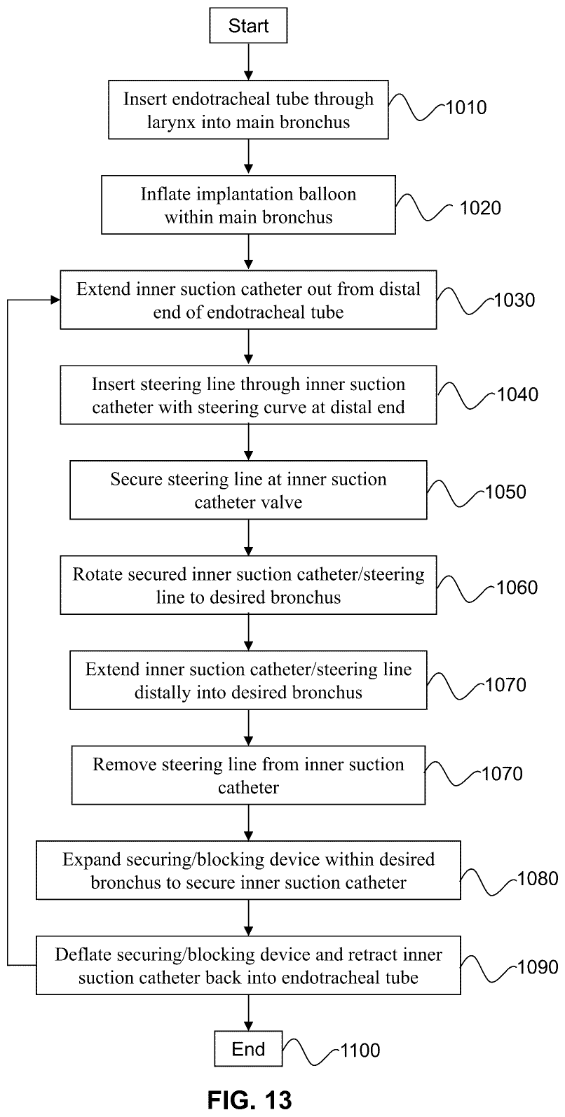

### DETAILED DESCRIPTION OF THE EMBODIMENTS

As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one skilled in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the features of the systems, apparatuses, and methods that are regarded as novel, it is believed that the systems, apparatuses, and methods will be better understood from a consideration of the following description in conjunction with the drawing figures, in which like reference numerals are carried forward.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Alternate embodiments may be devised without departing from the spirit or the scope of the invention. Additionally, well-known elements of exemplary embodiments of the systems, apparatuses, and methods will not be described in detail or will be omitted so as not to obscure the relevant details of the systems, apparatuses, and methods.

Before the systems, apparatuses, and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in tum, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider them as being the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Herein various embodiments of the systems, apparatuses, and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

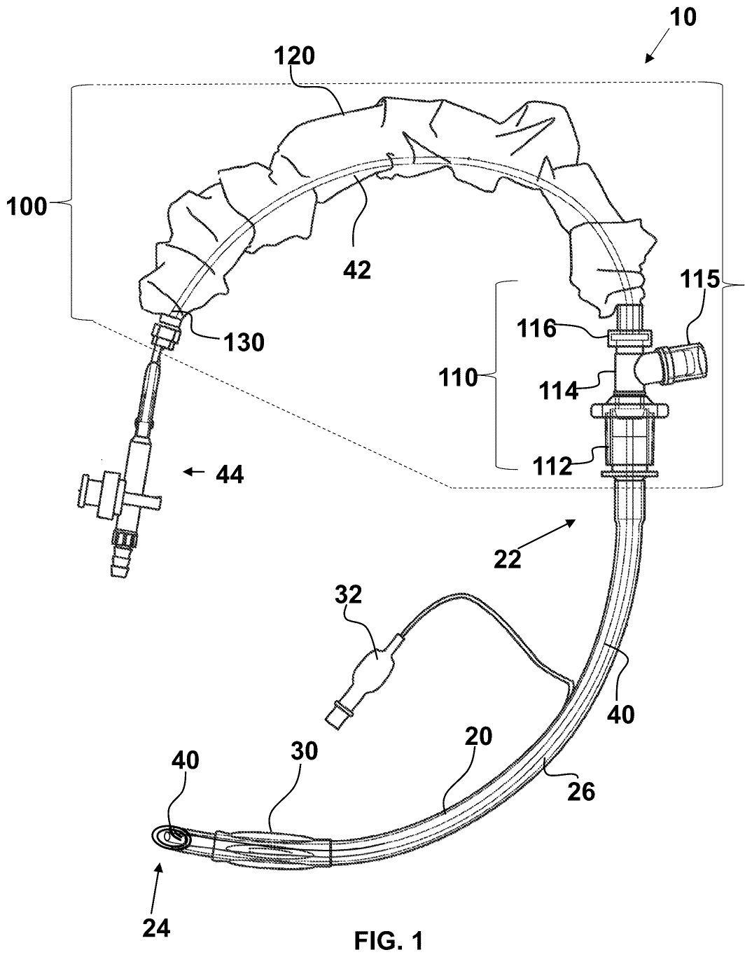

Described now are exemplary embodiments. Referring now to the figures of the drawings in detail and first, particularly to FIG. 1, there is shown a first exemplary embodiment of an endotracheal lung suction system 10 having an endotracheal tube 20 defining a tube lumen 26. The endotracheal tube 20 has an implantation balloon 30 adjacent a distal end 24 thereof, the balloon 30 being shown in a deflated state. An inner suction catheter 40 passes through the tube lumen 26 in a system installation position or initial state. The implantation balloon 30 of the endotracheal tube 20 is inflated through an inflation connector 32. The proximal tube end 22 of the endotracheal tube 20 is connected to a standard suction assembly or apparatus 100. The suction assembly 100 includes, at a distal end thereof, a ventilator connection subassembly 110 having a hollow endotracheal tube connector 112, a hollow ventilation junction 114, and a hollow tube entry port 116. The endotracheal tube connector 112 fluid tightly connects the interior tube lumen 26 of the endotracheal tube 20 to the interior of the ventilation junction 114. An input 115 of the ventilation junction 114 fluid-tightly connects to a non-illustrated breathing machine, which can be, for example, a mechanical, powered ventilator or a bag valve mask, sometimes known by the proprietary name AMBU® bag or generically as a manual resuscitator or “self-inflating bag.” The tube entry port 116 allows various devices to pass into and through the interior of the ventilation junction 114 and into and through the interior lumen of the endotracheal tube 20. Shown in the suction assembly 100 of FIGS. 1 through 9 is the proximal portion 42 of the hollow suction catheter 40 fluidically sealed off from the environment by a flexible bag 120 having a distal end fluidically sealed to the tube entry port 116, for example, by a fluid-tight seal around the periphery of the tube entry port 116. A proximal end of the flexible bag 120 is sealed off from the environment by, for example, a Tuohy-Borst adapter or valve 130, through which the proximal-most portion of the suction catheter 40 traverses. A ventilator lumen 140 is, therefore, defined as starting proximally at the opening of the valve 130, extending distally through the flexible bag 120, through the tube entry port 116, through the ventilation junction 114, and through the endotracheal tube connector 112 to fluidically connect to the tube lumen 26 starting at the proximal end 22 of the endotracheal tube 20.

Suction catheter 40 traverses through the valve 130 The inner suction catheter 40 has a proximal end at which is secured a valve or adapter 44 that permits a leak-free, fluidic connection from the environment to the interior lumen of the suction catheter 40. The valve 44 can be an on-off type device (such as a ball valve) or a gradual opening/closing type device (such as a screw valve). In order to extend the inner suction catheter 40 out from the distal tube end 24 of the endotracheal tube 20, the surgeon moves the valve 44 towards the tube entry port in a manner that compresses the flexible bag 120 longitudinally (friction is minimized and insertion is most efficient if the longitudinal axes of the valve 44 and the proximal end 42 of the inner suction catheter 40 are substantially aligned with the longitudinal axis of the tube entry port 116 during extension of the distal end of the inner suction catheter 20).

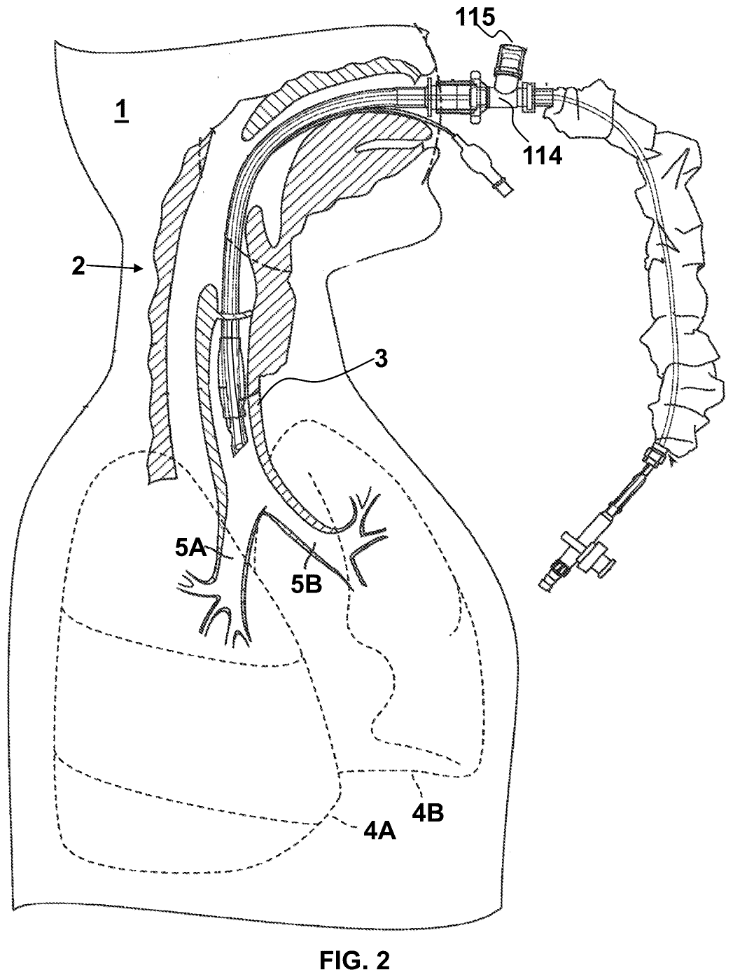

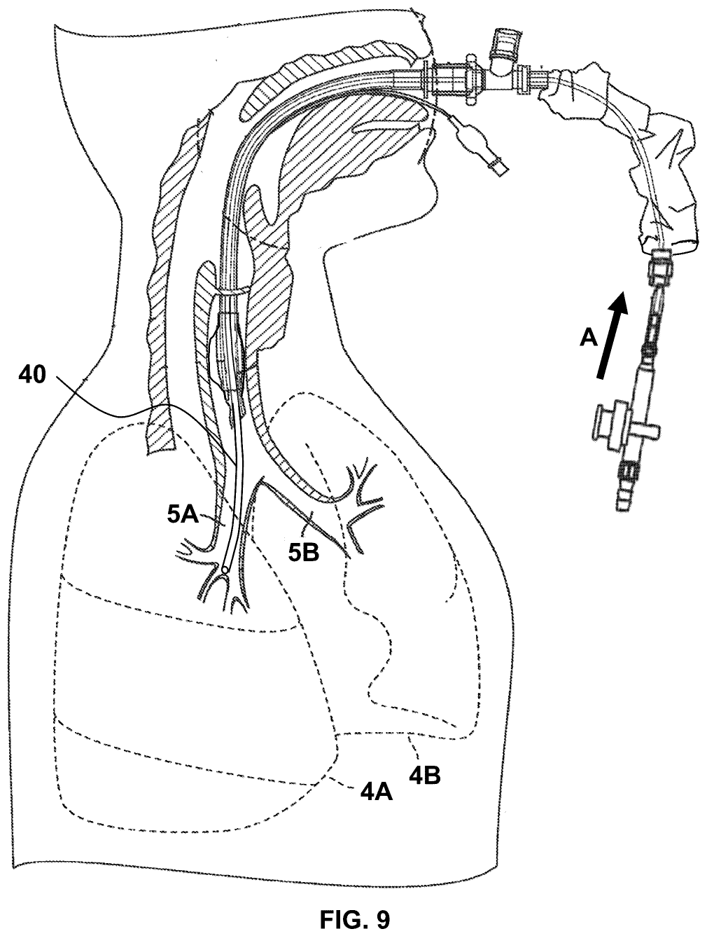

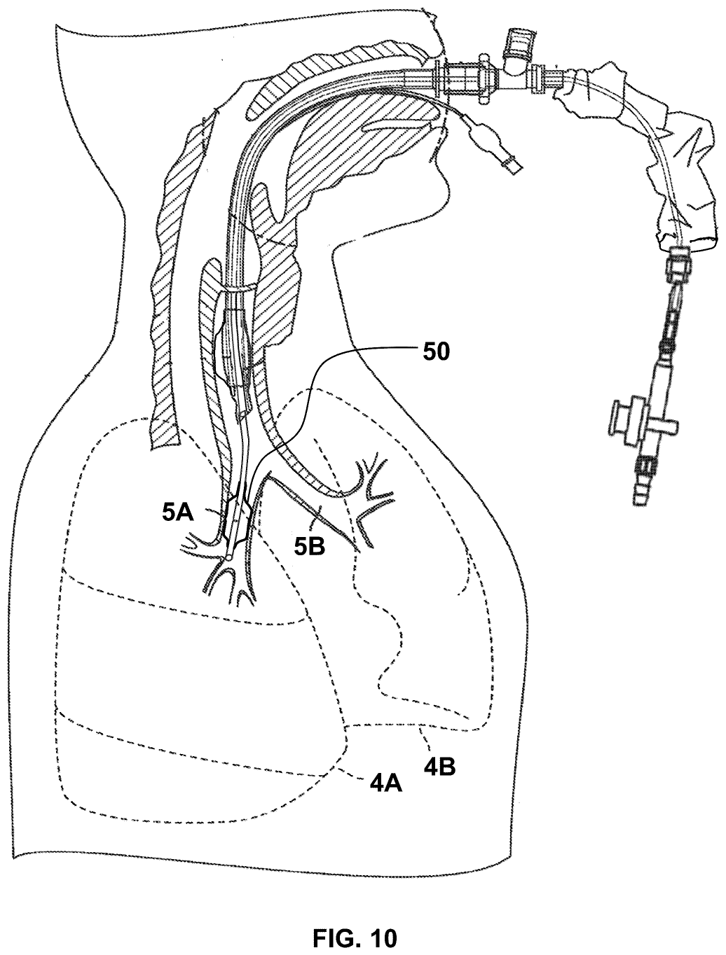

As first shown in detail in FIG. 9, a distal portion of the inner suction catheter 40 has a reverse angle that allows placement in the desired bronchus. The inner catheter securing and blocking device 50 is able, when expanded, to center the distal end of the inner suction catheter 40 in a lumen in which the distal end of the inner suction catheter 40 is placed. In a particular exemplary embodiment where the system 10 is used in a endotracheal procedure to access the interior of a patient's lungs, the inner catheter securing and blocking device 50 centers the distal end of the inner suction catheter 40 within the interior lumen of either the left or right main stems (also referred to as main bronchus) or, even further distally, in the right upper lobar bronchus, the right intermediate bronchus, the right middle lobar bronchus, the right lower lobar bronchus, the left upper lobar bronchus, or the left lower lobar bronchus. While in the system installation position or initial state, shown for example in FIGS. 1 and 2, the inner catheter securing and blocking device 50 rests in a contracted, reduced, or unexpanded state. A surgeon changes the inner catheter securing and blocking device 50 from the unexpanded state (shown in FIGS. 1 to 3 and 5 to 9) to the expanded state (shown in FIG. 10) when a surgeon decides to temporarily implant or secure the inner catheter securing and blocking device 50 in a lumen of the patient's lung 4A, 4B.

The drawings of FIGS. 2 through 10 and 13 illustrate methods and processes of how the novel inner suction catheter 40 and inner catheter securing and blocking device 50 operate. FIG. 2 illustrates (Step 1010) how an endotracheal tube 20 is inserted through a larynx 2 and into a main bronchus 3 of a patient 1. One exemplary method of determining that the distal end 24 of the endotracheal tube 20 is within the main bronchus 3 (as shown in FIG. 2) is to employ a manual or automatic resuscitator at the input 115 of the ventilation junction 114 and inflate the patient's lungs 4A (right), 4B (left). FIG. 2 shows the endotracheal tube 20 inserted through a patient's mouth, esophagus, trachea 2, and within the main bronchus 3 with an implantation balloon 30 of the endotracheal tube 20 in a deflated state. The inner suction catheter 40 is shown in a system installation position of the endotracheal tube, the suction catheter 40 being in an initial non-deployed state.

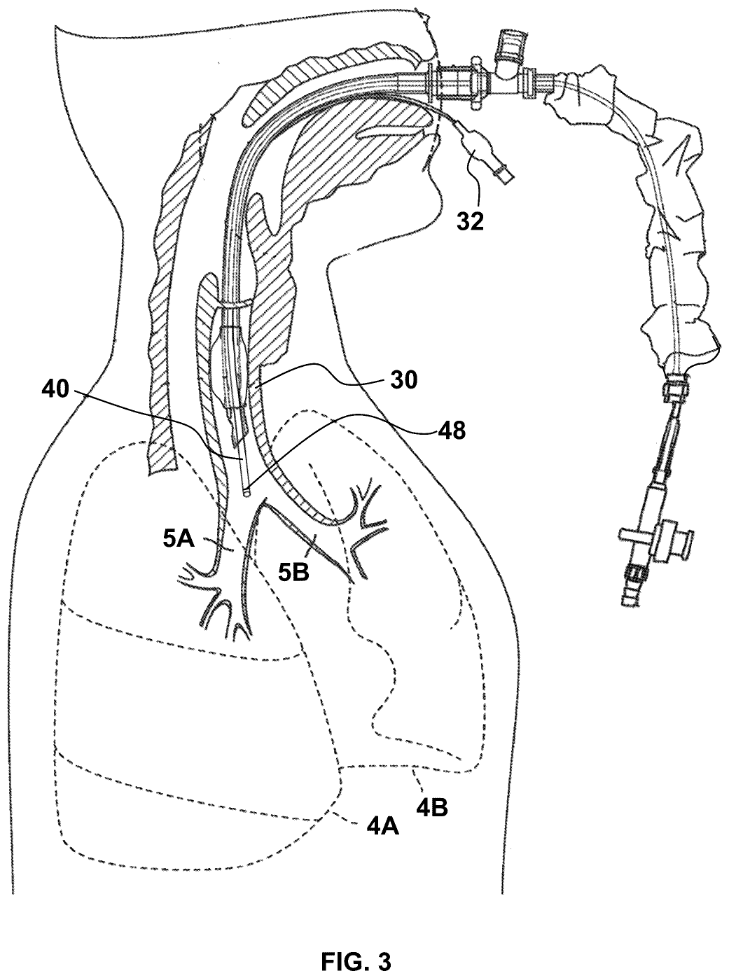

When correct implantation can be confirmed, the surgeon inflates (Step 1020) the implantation balloon 30 of the endotracheal tube 20 within the main bronchus 3 through the inflation connector 32, as shown in FIG. 3. Inflation of the implantation balloon 30 can occur with a liquid (saline, for example) or a gas (air, for example). In FIG. 3, the implantation balloon 30 of the endotracheal tube 20 is expanded within the patient's trachea 2 and is shown in an inflated state. As this exemplary embodiment of the implantation balloon 30 has a substantially circular cross-section, inflation substantially centers the implantation balloon 30 within the main bronchus 3, which, thereby, centers the inner lumen of the endotracheal tube 20 as well. When the distal end 24 of the endotracheal tube 20 is secured within the main bronchus 3 by the implantation balloon 30, the inner suction catheter 40 can be extended out from the distal end 24 of the endotracheal tube 20 (Step 1030). FIG. 3 illustrates a partial extension of the inner suction catheter 40 into the proximal portion of the right main stem bronchus 5A and is shown in a first deployed state. As used herein, the term “first” does not mean that it must be in order. This term is used to distinguish other deployed states of the inner suction catheter 40. The inner catheter securing and blocking device 50 of the inner suction catheter 40 is in an unexpanded state in FIGS. 2 and 3 and, therefore, cannot be seen in these two figures but is shown, for example, in FIG. 1.

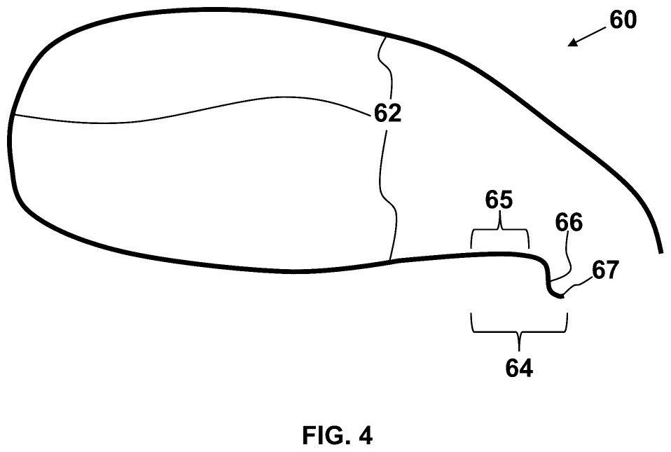

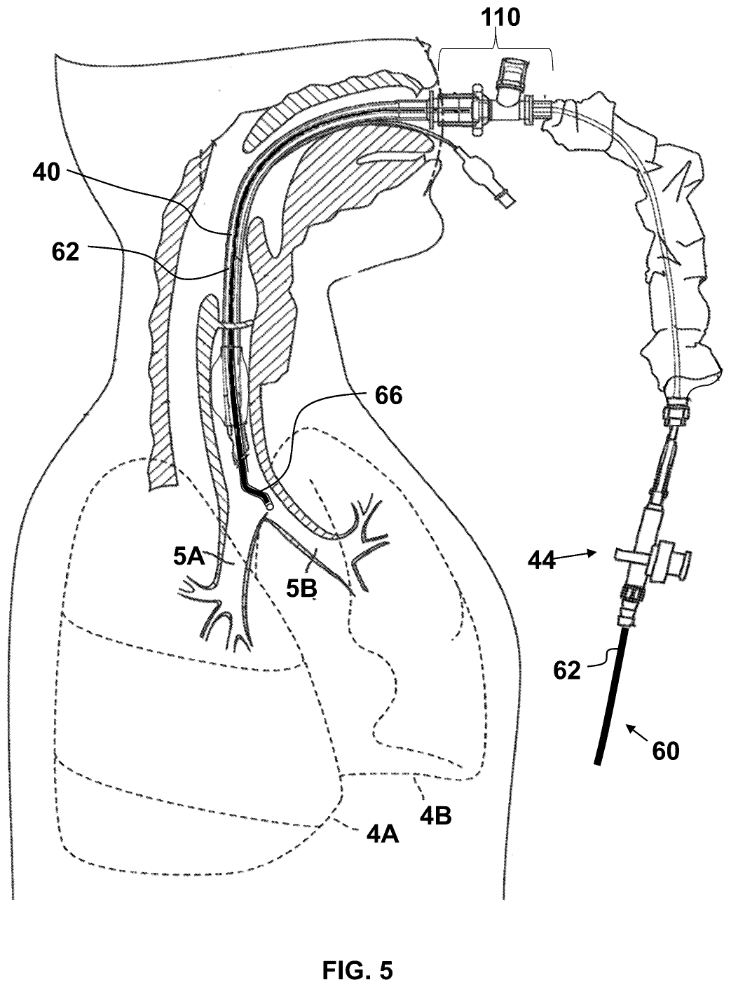

It is known that anatomy in most humans of the trachea and the right and left main bronchi are shaped such that the lumen of the trachea aligns more with the lumen of the right main bronchus than with the lumen of the left main bronchus. Therefore, without some guidance, any catheter extending from the distal end 24 of the endotracheal tube 20 will most likely enter right main bronchus 5A, more than 95% of the time. This is not to say that it will always enter the right main stem bronchus 5A. Therefore, a surgeon cannot be sure that extending the inner suction catheter 40 will enter the right main stem bronchus 5A, if that is what is desired. Conversely, a surgeon cannot be sure that extending the inner suction catheter 40 will not enter the left main stem bronchus 5B, if that is what is desired. To add a level of surety and reliability to the steering of the distal end 46 of the inner suction catheter 40, the present devices, methods, and processes include an inner catheter steering line 60. An exemplary embodiment of the inner catheter steering line 60 is shown in FIG. 4 as a solid rod having a diameter less than the inner diameter of the inner suction catheter 40 to be able to slide within the inner suction catheter 40. This embodiment has a proximal portion 62 that is flexible enough to be able to curve within the various curved portions of the inner suction catheter 40 and the endotracheal tube 20 without kinking or moving the inner suction catheter 40 too far from following the curvature of the endotracheal tube 20, which curve is depicted in FIG. 5. Therefore, depiction of the proximal portion 62 of the inner catheter steering line 60 in FIG. 4 is shown in a flexibly curved state even though the proximal portion 62 is substantially straight when left on a surface in a steady state. The distal portion of the inner catheter steering line 60, in comparison with the proximal portion 62 thereof, is set with a steering curve 64 in a pre-set shape, embodiments of which are explained in further detail below. The proximal portion 62 is, for example, 80% or more of the entire length, more particularly, 90% or more of the entire length, and, in particular, 95% or more of the entire length. The proximal portion 62 has a columnar stiffness to be able to be inserted within the inner suction catheter 40 (Step 1040) but still remain flexible enough to not force the inner suction catheter 40 out of its usable shape, which is shown in the various figures and, in particular, in FIG. 5. An exemplary material is a shape memory alloy, such as Nitinol, for example, in the shape of a rod.

The exemplary embodiment of the steering curve 64 shown in FIG. 4 and has an approximate S-bend shape. This S-bend is defined as having a proximal extent 65 set colinearly with the longitudinal extent of the proximal portion 62 of the inner catheter steering line 60. Distal of the proximal extent 65 is an intermediate extent 66 that curves away from the proximal extent 65 and has a short length extending at an angle to the immediately preceding proximal extent 65. Short, as defined here, is a distance that is less than a diameter of a smaller one of an inner diameter of the right and left main stem bronchi. The proximal extent 65, the first bend, and the intermediate extent 66 are pre-set, for example, with a shape memory material. The distal extent 67 of inner catheter steering line 60 is the portion distal of the intermediate extent 66 and is a length at an angle to the immediately preceding intermediate extent 66 to define a second bend at the distal end of the intermediate extent 66. The remaining distal extent 67 can be, but is not required to be, substantially parallel to a longitudinal direction of the proximal portion 65. Accordingly, when the inner catheter steering line 60 is inserted all the way to place the steering curve 64 at the distal catheter end 46 of the inner suction catheter 40 (depicted in FIG. 5), the pre-set shape of the steering curve 64 bends the distal end 46 of the inner suction catheter 40 substantially into the same S-bend shape of the steering curve (Step 1040). In the drawing of FIG. 5, the initial installation of the inner catheter steering line 60 bends the distal end 46 of the inner suction catheter to direct it into the left main bronchus 5B.

The inner catheter steering line 60 can be of various materials, however, one exemplary material that is particularly useful is nitinol because the inner catheter steering line 60 can be pre-set with differing shapes and, in particular, with a steering curve. Visual location of the steering curve 64 within a given one of the right or left main bronchus 5A, 5B (or neither) can be confirmed easily if the inner catheter steering line 60 is radiopaque. If the steering line 60 is, e.g., of nitinol, then the steering line 60 is naturally radiopaque. Likewise, visual confirmation of a location of the distal catheter end 46 of the inner suction catheter 40 having the steering curve 64 therein as well as a position of the distal end 46 of the inner suction catheter 40 with respect to the steering curve 64 can be made with a radiopaque marker 48 placed at the end of the inner suction catheter 40. One exemplary embodiment of a radiopaque marker 48 is shown in FIG. 3.

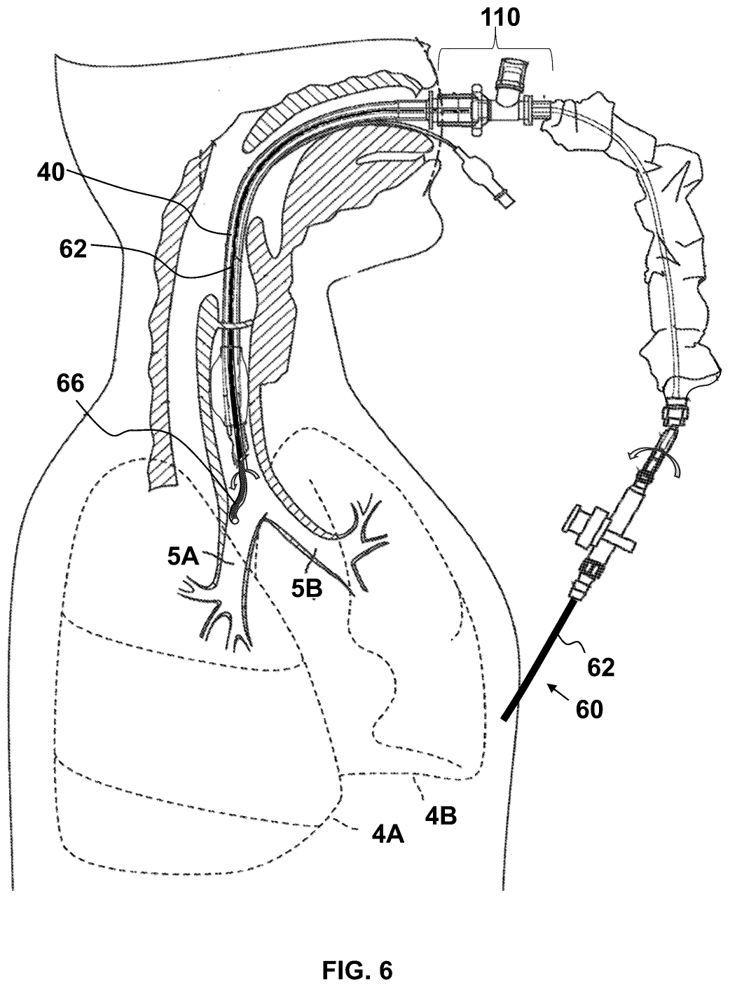

The steering line 60 is secured at the valve 44 of the inner suction catheter 40 (Step 1050). In an exemplary embodiment, the securement is both longitudinally (lengthwise) and radially (clockwise). This securement is accomplished either manually or mechanically. To manually secure the steering line 60 to the valve 44, the surgeon grasps both the steering line 60 and the valve 44. Longitudinal distal movement of the inner suction catheter 40 occurs by sliding both the steering line 60 and the inner suction catheter 40 distally through the ventilator connection subassembly 110. Rotational movement of the inner suction catheter 40 to guide the distal catheter end 46 into a desired left or right main stem bronchus 5B, 5A occurs by rotating at least the steering line 60 but also both the steering line 60 and the inner suction catheter 40 with respect to the ventilator connection subassembly 110. Torsional stiffness of the steering line 60 permits rotation of the distal catheter end 46 while it is present in the main bronchus 3. This rotation is depicted by the transition from the view of FIG. 5 to the view of FIG. 6 (Step 1060). In particular, FIG. 5 illustrates the inner catheter steering line 60 in the deployed state within the inner suction catheter 40 and positioned to place the distal end 46 of the inner suction catheter 40 within the entrance of the left main bronchus 5B of a patient's lung 4B. Here, the inner catheter securing and blocking device 60 still remains in the unexpanded state and the implantation balloon 30 of the endotracheal tube 20 is expanded within the trachea 2 in the inflated state. FIG. 6 illustrates the distal end 46 of the inner suction catheter 40 oriented to enter the right main stem bronchus 5A after the valve 44 has been rotated, for example, through 180 degrees or ore. As the torsional stiffness of the steering line 60 is not ideal, rotation of the proximal end of the steering line 60 (either at or proximal of the valve 44) will not exactly match a rotation of the distal steering curve 64. Therefore, it is envisioned that a surgeon may have to rotate the steering line 60 more than 180 degrees to obtain a 180 degree rotation of the steering curve 64. Like FIG. 5, the illustration of FIG. 6 shows the inner catheter securing and blocking device 60 in the unexpanded state and the implantation balloon 30 expanded within the trachea 2 in the inflated state.

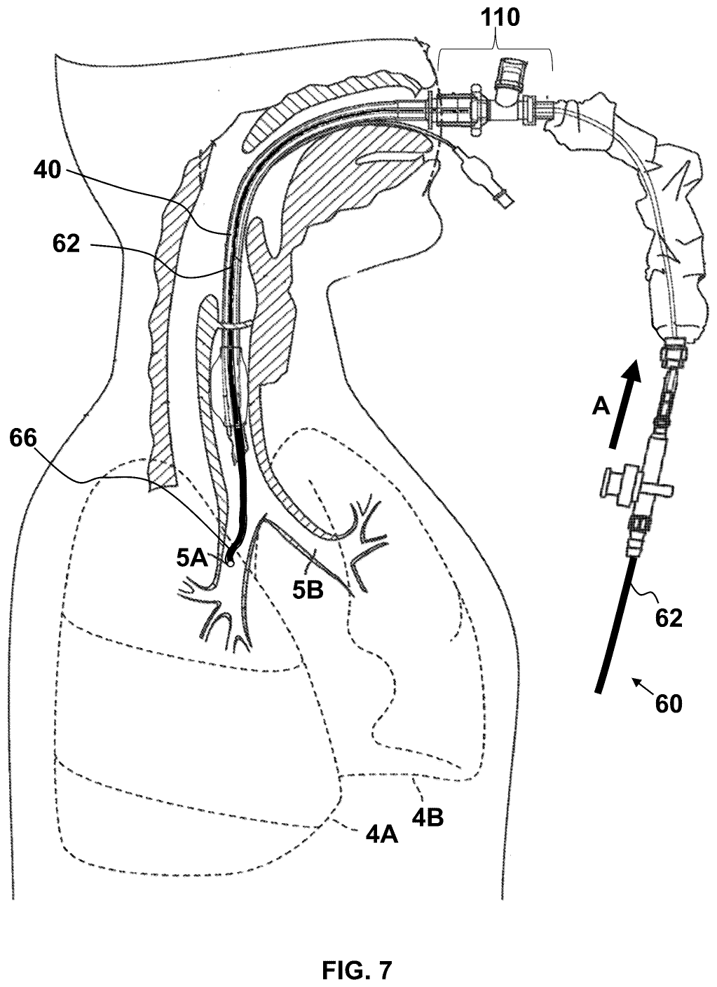

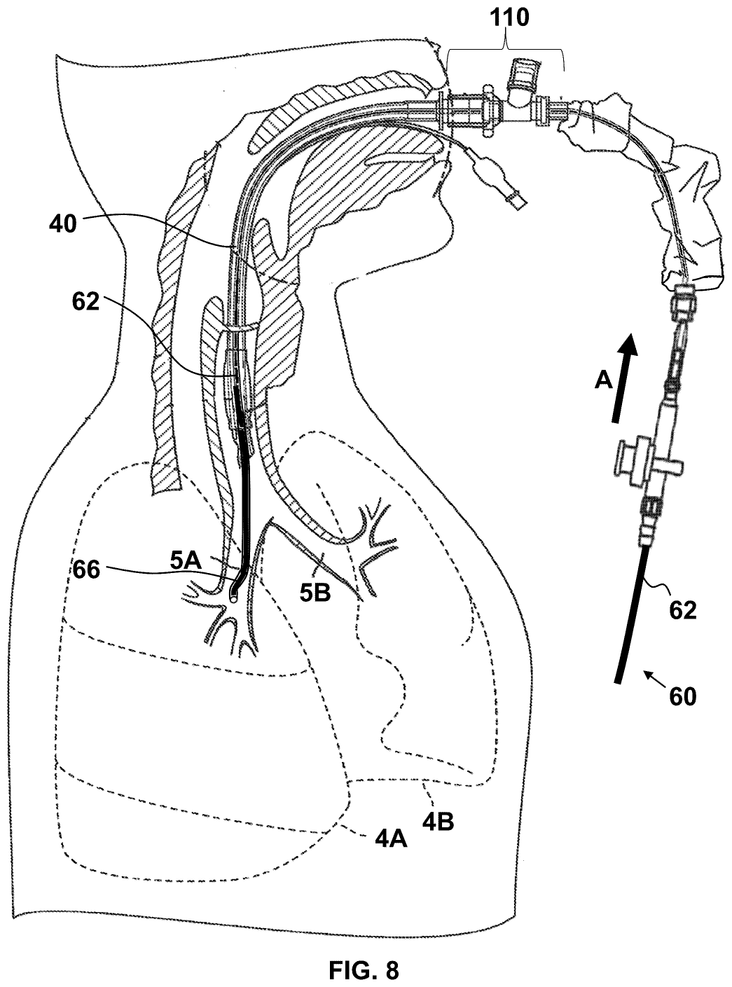

The transition from the view of FIG. 6 to FIG. 7 to FIG. 8 shows the surgeon distally inserting (arrow A) the inner catheter steering line 60 and the inner suction catheter 40 together into the ventilator connection subassembly 110 (Step 1070). FIG. 7 illustrates the inner suction catheter 40 in a second deployed state further in the right main stem bronchus 5A of the patient's right lung 5A (Step 1070). The inner catheter securing and blocking device 60 is in the unexpanded state. FIG. 8 illustrates the inner suction catheter 40 in a third deployed state further in the right main stem bronchus 5A of the patient's right lung 5A (Step 1070). The inner catheter securing and blocking device 60 is in the unexpanded state.

When the inner suction catheter 40 is in the desired final extended state, the surgeon removes the inner catheter steering line 60 from the inner suction catheter 40 (Step 1080). Removal of the steering curve 64 from the inner suction catheter 40 allows the distal end 46 of the inner suction catheter 60 to return back to its steady state. This condition is shown in the view of FIG. 9, with the inner catheter securing and blocking device 50 in the unexpanded state.

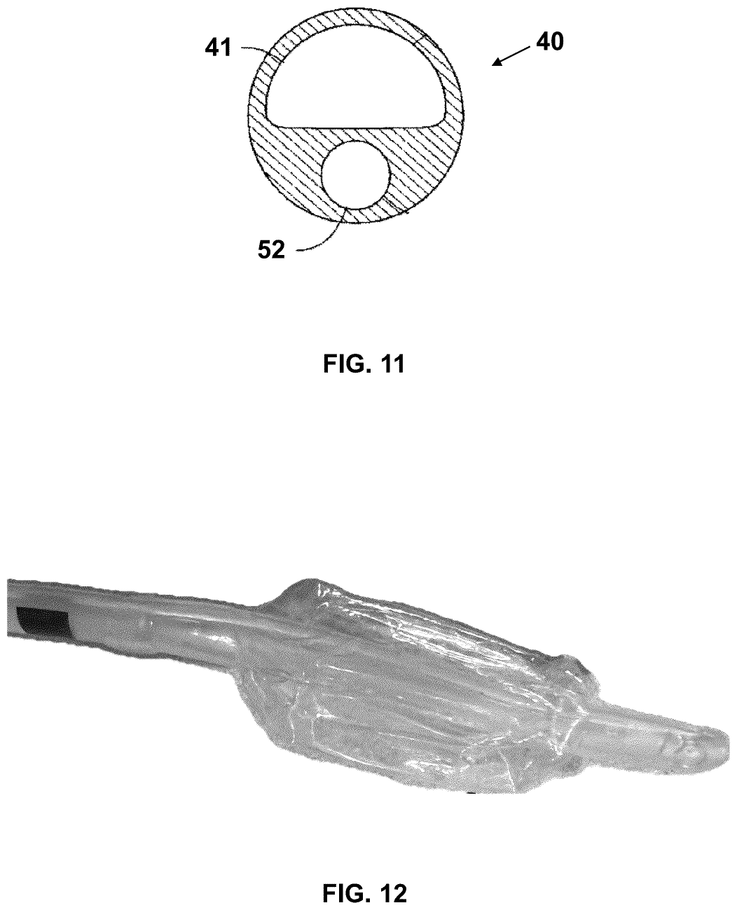

At this point in the procedure, the surgeon desires to secure the inner suction catheter 40 within the right primary bronchus 5A (or even further within the lung 4A). To affect this securement, the inner catheter securing and blocking device 50 is transitioned into an expanded state (Step 1080) shown in FIG. 10. In an exemplary embodiment, the inner catheter securing and blocking device 50 is a catheter balloon similar to the implantation balloon 30 of the endotracheal tube 20. As it secures a smaller and lighter tube within a lung lumen, the inner catheter securing and blocking device 50 can be smaller and lighter than the implantation balloon 30 of the endotracheal tube 20. Inflation of the inner catheter securing and blocking device 50 occurs through an inflation lumen that is colinear with the central lumen of the inner suction catheter 40. This inflation lumen is not illustrated in the views of FIGS. 1 to 3 or 5 through 9. However, a cross-section of an exemplary embodiment of the inner suction catheter 50 is shown in FIG. 11 and depicts a main lumen 41 and a securing catheter inflation lumen 52. After aspiration, the inner catheter securing and blocking device 50 is deflated and the inner suction catheter 40 is retracted back into the endotracheal tube 20 (Step 1090). The procedure can be repeated (e.g., Steps 1030 to 1090) either for the same bronchus or the other bronchus.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all the other exemplary embodiments described herein and in any combination, grouping, or arrangement. Use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The foregoing description and accompanying drawings illustrate the principles, exemplary embodiments, and modes of operation of the systems, apparatuses, and methods. However, the systems, apparatuses, and methods should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the systems, apparatuses, and methods as defined by the following claims.

1. An endotracheal lung suction system, comprising:
an endotracheal tube defining a tube lumen and comprising:
a distal tube end defining a distal opening of the tube lumen;
a proximal tube end defining a proximal opening of the tube lumen;
an inflation connector; and
an implantation balloon fluidically connected to the inflation connection and through which the implantation balloon is inflated, the implantation balloon disposed adjacent the distal tube end;
a suction assembly comprising:
a distal endotracheal tube connector at the proximal tube end and defining a tube opening fluidically connected to an interior of the tube lumen through the distal opening of the tube lumen; and
a proximal valve defining a proximal valve catheter opening fluidically connected through the suction assembly to the interior of the tube lumen; and
an inner suction catheter shaped and sized to pass through and slide within the suction assembly from proximal of the proximal valve, distally through the tube opening of the endotracheal tube connector, and through the tube lumen to extend slidably out from and back into the proximal opening of the distal tube end, the inner suction catheter comprising:
a proximal portion comprising a proximal catheter end;
a distal portion comprising a distal catheter end;
an inflatable, hollow, inner-suction-catheter securing device at the distal portion and comprising an inflation interior defining an inflation interior opening; and
a catheter body defining:
a main lumen extending through the inner suction catheter from the proximal catheter end to the distal catheter end; and
a catheter-securing inflation lumen extending through the inner suction catheter parallel to the main lumen from the proximal portion distally to the inflation interior opening.

2. The system according to claim 1, which further comprises an inner catheter steering line shaped and sized to pass through and slide within the main lumen of the inner suction catheter, the inner catheter steering line comprising a distal portion with a steering curve.

3. The system according to claim 2, wherein:
the steering curve comprises a given shape; and
at least the distal catheter end of the inner suction catheter is flexible such that placement of the steering curve within the distal end causes the distal catheter end to take approximately the given shape of the steering curve therewithin.

4. The system according to claim 3, wherein the given shape is an S-bend.

5. The system according to claim 2, wherein:
the steering curve comprises a given shape; and
the inner suction catheter is flexible to permit the given shape of the steering curve to slide through the main lumen from the proximal catheter end to the distal catheter end.

6. The system according to claim 5, wherein:
the given shape is an S-bend; and
the inner suction catheter is flexible to permit the S-bend of the steering curve to slide through the main lumen from the proximal catheter end to the distal catheter end and, responsive to the steering curve being placed in the distal catheter end, the distal catheter end takes approximately the S-bend shape.

7. The system according to claim 1, wherein the distal portion at the distal catheter end is curved.

8. The system according to claim 1, wherein the distal portion at the distal catheter end has an S-shape.

9. The system according to claim 1, wherein the inner suction catheter further comprises a valve at the proximal portion, the valve permitting a leak-free, fluidic connection from the environment to the interior of the main lumen of the inner suction catheter.
